(19) United States
(12) Patent Application Publication
(10) Pub. No.: US 2020/0139016 A1
(43) Pub. Date: May 7, 2020

(54) ELASTOMER MOLDED BODY FOR MEDICAL DEVICE, METHOD OF MANUFACTURING THEREOF, AND MEDICAL DEVICE

(71) Applicant: OLYMPUS CORPORATION, Tokyo (JP)

(72) Inventor: Kazuhito TOMIZUKA, Yokohama-shi (JP)

(73) Assignee: OLYMPUS CORPORATION, Tokyo (JP)

(21) Appl. No.: 16/713,705

(22) Filed: Dec. 13, 2019

Related U.S. Application Data
(63) Continuation of application No. PCT/JP2018/021403, filed on Jun. 4, 2018.

(30) Foreign Application Priority Data
Jun. 21, 2017 (JP) 2017-121690

(51) Int. Cl. A61L 29/12; B29B 7/90; C08K 3/36

(52) U.S. Cl. CPC A61L 29/126; B29K 2509/02; C08K 3/36; B29B 7/90

(57) ABSTRACT

An elastomer molded body for a medical device includes an elastomer portion and a plurality of silica particles. The elastomer portion contains a fluorine-based elastomer. The plurality of silica particles are more densely distributed in outside of a center portion of the elastomer portion than inside of the center portion, such that at least some of the plurality of silica is exposed to a surface of the elastomer portion.

The application is a continuation application based on a PCT Patent Application No. PCT/JP2018/021403, filed Jun. 4, 2018, whose priority is claimed on Japanese Patent Application No. 2017-121690, filed Jun. 21, 2017. The content of both the PCT Application and the Japanese Application are incorporated herein by reference.

### BACKGROUND OF THE INVENTION

### Field of the Invention

The present invention relates to an elastomer molded body for a medical device, a method of manufacturing of an elastomer molded body for a medical device, and a medical device.

### Description of Related Art

For example, regarding a covering member for a medical device which covers a surface of a medical device such as an endoscope, an elastomer molded body having tolerance under a disinfection and sterilization environment is used. Fluorine rubber is known as a material of such an elastomer molded body.

For example, Japanese Unexamined Patent Application, First Publication No. H5-300938 discloses a rubber tube for a bending portion of an endoscope. The tube was formed by vulcanization molding a blended and kneaded material containing, in proportions by weight, 10 to 30 parts of liquid fluorine rubber, 0.1 to 1.5 parts of Perhexa (registered trademark) 25B, 0.3 to 4 parts of triallyl isocyanate, and 1 to 10 parts of reinforcing carbon having an average particle size of 150 mμ or less with respect to 100 parts of a ternary copolymer of vinylidene fluoride-hexafluoropropylene-tetrafluoroethylene.

### SUMMARY OF THE INVENTION

An elastomer molded body for a medical device according to a first aspect of the present invention includes an elastomer portion containing a fluorine-based elastomer; and a plurality of silica particles which are more densely distributed in outside of a center portion of the elastomer portion than inside of the center portion and of which at least some are exposed to a surface of the elastomer portion.

A medical device according to a second aspect of the present invention includes the elastomer molded body for a medical device.

A method of manufacturing of an elastomer molded body for a medical device according to a third aspect of the present invention includes kneading an elastomer molding material containing a crosslinkable first fluorine-based elastomer, a second fluorine-based elastomer composed of a liquid fluorine-based elastomer that is not to crosslink with the first fluorine-based elastomer, a polymer oil, and a plurality of silica particles, thus forming a kneaded material; molding the kneaded material using a mold; and heating the molded kneaded material to a temperature that is equal to or higher than a boiling point of the polymer oil to crosslink the first fluorine-based elastomer in the kneaded material.

### BRIEF DESCRIPTION OF THE DRAWINGS

FIG. 1 is a cross-sectional view schematically showing an example of an elastomer molded body for a medical device according to a first embodiment of the present invention.

FIG. 2 is a partially enlarged view of the part A in FIG. 1.

FIG. 3 is a cross-sectional view schematically showing an example in which silica particles are distributed on a surface layer portion of the elastomer molded body for a medical device according to the first embodiment of the present invention.

FIG. 4A is a schematic view showing an example of a manufacturing process of an elastomer molded body for a medical device according to the first embodiment of the present invention.

FIG. 4B is a schematic view showing an example of a manufacturing process of an elastomer molded body for a medical device according to the first embodiment of the present invention.

FIG. 4C is a schematic view showing an example of a manufacturing process of an elastomer molded body for a medical device according to the first embodiment of the present invention.

FIG. 5A is a schematic view showing an example of a manufacturing process of an elastomer molded body for a medical device according to the first embodiment of the present invention.

FIG. 5B is a schematic view showing an example of a manufacturing process of an elastomer molded body for a medical device according to the first embodiment of the present invention.

FIG. 5C is a schematic view showing an example of a manufacturing process of an elastomer molded body for a medical device according to the first embodiment of the present invention.

FIG. 6 is a perspective view schematically showing an example of a medical device according to a second embodiment of the present invention.

### DETAILED DESCRIPTION OF THE INVENTION

In the following, embodiments of the present invention will be described with reference to the drawings. In all the drawings, the same or corresponding members are denoted with the same reference numerals in different embodiments, and common descriptions are omitted.

### First Embodiment

An elastomer molded body for a medical device according to a first embodiment of the present invention will be described.

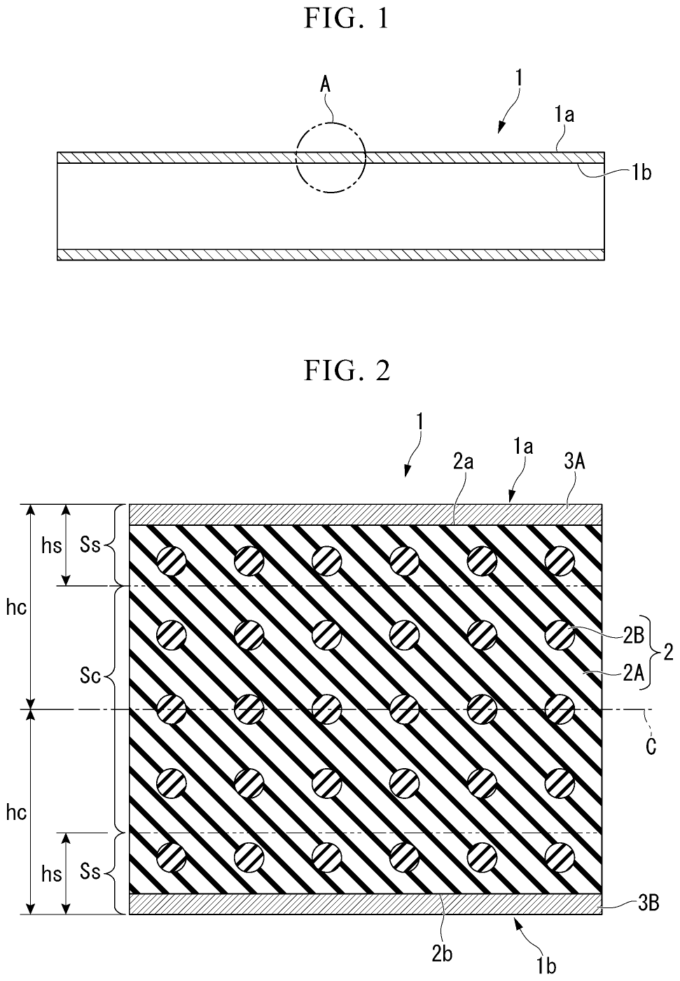

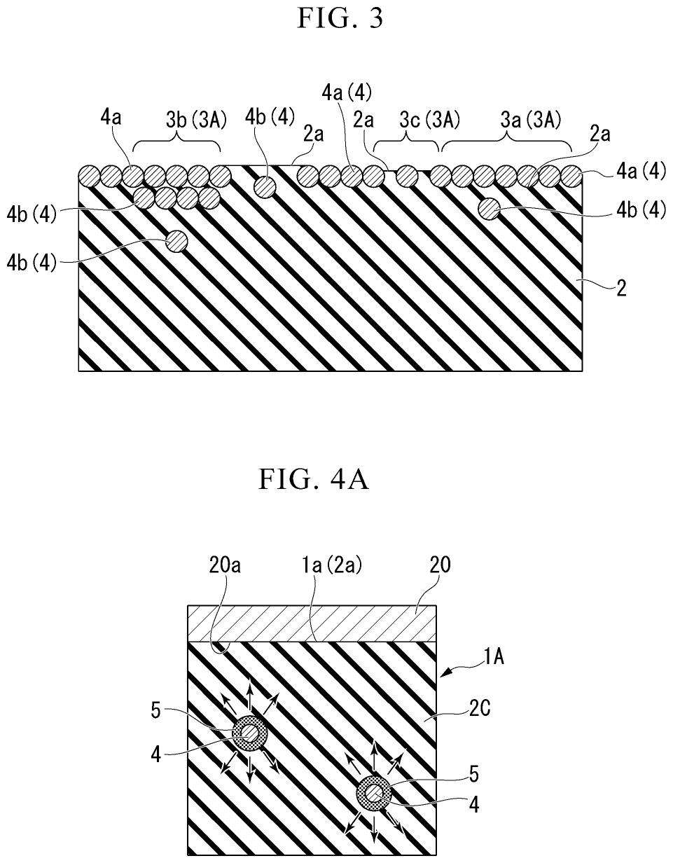

FIG. 1 is a cross-sectional view schematically showing an example of the elastomer molded body for a medical device according to the first embodiment of the present invention. FIG. 2 is a partially enlarged view of the part A in FIG. 1. FIG. 3 is a cross-sectional view schematically showing an example in which silica particles are distributed on a surface layer portion of the elastomer molded body for a medical device according to the first embodiment of the present invention.

The medical device in which the elastomer molded body for a medical device according to the present embodiment is used is not particularly limited. Examples of medical devices in which the elastomer molded body for a medical device according to the present embodiment can be used include an endoscope and a surgical treatment instrument.

When the elastomer molded body for a medical device according to the present embodiment is used for an endoscope, the elastomer molded body for a medical device may be used for, for example, an outer cover of a bending portion or an insertion portion, a fold-proof member for reinforcing a tubular member, a switch button, an outer cover that covers a switch button, an O-ring, and a sealing member.

The shape of the mold for the elastomer molded body for a medical device according to the present embodiment is not particularly limited. The shape of the elastomer molded body for a medical device is determined according to the needs of the medical device in which the elastomer molded body for a medical device is used.

Examples of the shape of the elastomer molded body for a medical device include a sheet shape, a rod shape, a ring shape, a tubular shape, a box shape, a cap shape, a coil shape, a bag shape, a band shape, and a block shape. For example, regarding the shape of the elastomer molded body for a medical device, an appropriate three-dimensional shape which cannot be simplified as in the above shapes may be used.

In the following, as shown in FIG. 1, an example in which the shape of the elastomer molded body for a medical device is a cylindrical shape will be described.

A medical device tube 1 as an elastomer molded body for a medical device according to the present embodiment is formed in a cylindrical shape. The cross-sectional shapes of an outer circumferential surface 1a and an inner circumferential surface 1b of the medical device tube 1 are circular.

The medical device tube 1 may be used as a part of a medical device or the medical device tube 1 itself may be used as a medical device.

For example, the medical device tube 1 may be used as an outer tube of a bending portion or an insertion portion of an endoscope. For example, the medical device tube 1 may be used as a part of a medical device or a medical device, in order to form a flow path for an appropriate liquid or gas.

As shown in FIG. 2, the medical device tube 1 includes an elastomer layer 2 (elastomer portion) and surface silica layers 3A and 3B (silica particle group).

The elastomer layer 2 includes a crosslinked fluorine-based elastomer 2A (fluorine-based elastomer) and a liquid fluorine-based elastomer 2B (fluorine-based elastomer) dispersed in the crosslinked fluorine-based elastomer 2A.

Although not shown, the elastomer layer 2 may contain appropriate additive components as necessary. Examples of additive components include a crosslinking agent, a co-crosslinking agent, a filler, a tackifier, a processing aid, a curing agent, an antioxidant, and an acid acceptor. The additive components contained in the elastomer layer 2 may be of one type or of two or more types.

The crosslinked fluorine-based elastomer 2A is formed by crosslinking a polymeric fluorine compound.

Regarding a polymeric fluorine compound for forming the crosslinked fluorine-based elastomer 2A, for example, at least one of a binary copolymer and a ternary copolymer may be used.

Examples of binary copolymers include a vinylidene fluoride-hexafluoropropylene copolymer, a tetrafluoroethylene-propylene copolymer, a tetrafluoroethylene-fluoromethyl vinyl ether copolymer, and a tetrafluoro ethylene-ethylene copolymer.

Examples of ternary copolymers include a vinylidene fluoride-hexafluoropropylene-tetrafluoroethylene copolymer, a vinylidene fluoride-propylene-tetrafluoroethylene copolymer, and a vinylidene fluoride-tetrafluoroethylene-perfluoroalkyl vinyl ether copolymer.

A vinylidene fluoride-hexafluoropropylene-tetrafluoroethylene copolymer may be contained in the crosslinked fluorine-based elastomer 2A. When the crosslinked fluorine-based elastomer 2A contains a vinylidene fluoride-hexafluoropropylene-tetrafluoroethylene copolymer, crystallinity of the crosslinked fluorine-based elastomer 2A is further reduced. Therefore, the flexibility of the crosslinked fluorine-based elastomer 2A is further improved.

In order to adjust the flexibility of the elastomer layer 2, the liquid fluorine-based elastomer 2B is dispersed in the crosslinked fluorine-based elastomer 2A.

The liquid fluorine-based elastomer 2B has variation in molecular weight. Therefore, the size and shape of the liquid fluorine-based elastomer 2B vary depending on the molecular weight. However, according to kneading during production to be described below, distribution of the liquid fluorine-based elastomer 2B in the elastomer layer 2 is substantially uniform in the layer thickness direction and the longitudinal direction of the elastomer layer 2.

The liquid fluorine-based elastomer 2B is not crosslinked with the crosslinked fluorine-based elastomer 2A.

The liquid fluorine-based elastomer 2B is not particularly limited as long as it is a liquid at room temperature and can adjust the flexibility of the elastomer layer 2. Regarding the liquid fluorine-based elastomer 2B, an appropriate liquid fluorine-based elastomer not having crosslinking reactive group that forms a crosslinked structure of the liquid fluorine-based elastomer 2B is used.

The liquid fluorine-based elastomer 2B may be a material having favorable compatibility with raw materials of the crosslinked fluorine-based elastomer 2A. The number-average molecular weight of the liquid fluorine-based elastomer 2B may be 5,000 or less.

The liquid fluorine-based elastomer 2B is included in the elastomer layer 2 as necessary in order to make the elastomer layer be flexible. For example, 10 parts by mass or more and 30 parts by mass or less of the liquid fluorine-based elastomer 2B may be contained with respect to 100 parts by mass of the crosslinked fluorine-based elastomer 2A. For example, 10 parts by mass or more and 20 parts by mass or less of the liquid fluorine-based elastomer 2B may be contained with respect to 100 parts by mass of the crosslinked fluorine-based elastomer 2A.

The surface silica layers 3A and 3B are formed by a collection (silica particle group) of a plurality of silica particles (not shown in FIG. 2). The surface silica layers 3A and 3B are exposed from an outer circumferential surface 2a and an inner circumferential surface 2b of the elastomer layer 2 to form at least of the outer circumferential surface 1a and the inner circumferential surface 1b of the medical device tube 1.

The surface silica layers 3A and 3B may be thinner than the elastomer layer 2 so that the flexibility of the medical device tube 1 is not impaired. For example, the layer thickness of the surface silica layers 3A and 3B may be larger than 0 μm and 20 μm or less. For example, the layer thickness of the surface silica layers 3A and 3B may be larger than 0 μm and 10 μm or less.

Since the surface silica layers 3A and 3B are different only in their disposition, the configuration of the surface silica layer 3A will be mainly described below. Unless otherwise noted, the following description of the surface silica layer 3A similarly applies to the surface silica layer 3B.

FIG. 2 is a schematic view, and in FIG. 2, the surface silica layer 3A (3B) is shown as a layer that covers the entire outer circumferential surface 2a (the inner circumferential surface 2b) (surface) of the elastomer layer 2. However, the distribution of silica particles in the surface silica layer 3A is not limited to a uniform layer as shown.

FIG. 3 schematically shows various configuration examples of the surface silica layer 3A.

As shown in FIG. 3, a plurality of silica particles 4 are contained in the medical device tube 1. The plurality of silica particles 4 are classified into surface-exposed silica particles 4a and internal silica particles 4b.

The surface-exposed silica particles 4a are the silica particles 4 each of which is exposed at least a part thereof to the outside of the outer circumferential surface 2a of the elastomer layer 2. The surface-exposed silica particles 4a form at least a part of the outer circumferential surface 1a of the medical device tube 1.

The internal silica particles 4b are the silica particles 4 disposed inside the outer circumferential surface 1a of the medical device tube 1.

For example, the surface silica layer 3A may include a single layer dense silica layer 3a (silica particle group) in which the surface-exposed silica particles 4a cover the outer circumferential surface 2a, and are densely distributed on the outer circumferential surface 2a. The single layer dense silica layer 3a may cover the entire outer circumferential surface 2a or may cover a part of the outer circumferential surface 2a in an island shape. When the single layer dense silica layer 3a has an island shape, the outer periphery of the single layer dense silica layer 3a is in contact with the outer circumferential surface 2a constituting a part of the outer circumferential surface 1a.

For example, the surface silica layer 3A may include a multi-layered dense silica layer 3b (silica particle group) in which the surface-exposed silica particles 4a and one or more layers of the internal silica particles 4b stacked on the surface-exposed silica particles 4a cover the outer circumferential surface 2a, and are densely distributed on the outer circumferential surface 2a. The multi-layered dense silica layer 3b may cover the entire outer circumferential surface 2a or may cover a part of the outer circumferential surface 2a in an island shape. When the multi-layered dense silica layer 3b has an island shape, the outer periphery of the multi-layered dense silica layer 3b is in contact with the outer circumferential surface 2a constituting a part of the outer circumferential surface 1a.

For example, the surface silica layer 3A may include a dispersedly-distributed silica layer 3c (silica particle group) in which the surface-exposed silica particles 4a are distributed on the outer circumferential surface 2a with gaps therebetween in the axial direction and the circumferential direction. In the dispersedly-distributed silica layer 3c, the outer circumferential surface 2a is exposed between adjacent surface-exposed silica particles 4a in the outer circumferential surface 2a.

In the surface silica layer 3A, the single layer dense silica layer 3a, the multi-layered dense silica layer 3b, and the dispersedly-distributed silica layer 3c described above may be mixed in appropriate proportions.

The internal silica particles 4b in the medical device tube 1 may constitute a part of the surface silica layer 3A as in the above multi-layered dense silica layer 3b. All of the internal silica particles 4b may constitute a part of the surface silica layers 3A and 3B. That is, a configuration in which no internal silica particles 4b are contained in the elastomer layer 2 between the surface silica layers 3A and 3B may be used.

The internal silica particles 4b may be distributed to be spaced inward from the surface-exposed silica particles 4a and the outer circumferential surface 2a. In this case, the internal silica particles 4b do not constitute the surface silica layer 3A. However, even if the internal silica particles 4b not to constitute the surface silica layer 3A are contained in the elastomer layer 2, in the medical device tube 1, the silica particles 4 including the internal silica particles 4b are more densely distributed in outside of a center portion Sc of the elastomer layer 2 than inside of the center portion Sc.

As shown in FIG. 2, a distance from the outer circumferential surface 1a (the inner circumferential surface 1b) of the medical device tube 1 to a layer thickness center plane C that is the center of the elastomer layer 2 in the layer thickness direction is represented as hc.

In this case, a surface layer portion Ss of the medical device tube 1 is defined as being in a distance range of hs (=(⅓)·hc) from the outer circumferential surface 1a (the inner circumferential surface 1b). However, the distance hs is larger than the layer thickness of the surface silica layer 3A (3B).

The center portion Sc of the elastomer layer 2 is defined as an area excluding a range overlapping the surface layer portion Ss in the elastomer layer 2.

When it is described that the silica particles 4 are “more densely distributed in outside of the center portion Sc than inside of the center portion Sc,” it means that an amount of the silica particles 4 is larger in the surface layer portion Ss than in the center portion Sc.

For example, in the medical device tube 1, 0% or more and less than 20% of the silica particles 4 may be distributed in the center portion Sc, and 80% or more and 100% or less thereof may be distributed in the surface layer portion Ss. In the medical device tube 1, 0% or more and less than 10% of the silica particles 4 may be distributed in the center portion Sc, and 90% or more and 100% or less thereof may be distributed in the surface layer portion Ss. 100% of the silica particles 4 may be distributed in the surface layer portion Ss.

An amount of the silica particles 4 distributed in the center portion Sc and the surface layer portion Ss can be measured by, for example, counting the silica particles 4 in an appropriate cross section of the medical device tube 1.

When the elastomer molded body for a medical device is formed in a shape different from a cylindrical shape, such as a rod shape or a block shape, the surface layer portion and the center portion are distinguished in the same manner as in the distance hs based on a distance between the surface of the elastomer molded body for a medical device and the center of the elastomer molded body for a medical device in place of the distance hc.

As described above, in the medical device tube 1, the surface silica layer 3A (3B) with at least some of the plurality of silica particles 4 is located on the outer circumferential surface 2a (the inner circumferential surface 2b) of the elastomer layer 2. A distribution density of the plurality of silica particles 4 in the surface silica layer 3A (3B) provided on the outer circumferential surface 2a (the inner circumferential surface 2b) is higher than in a center portion of the elastomer layer 2 in the layer thickness direction.

Regarding a distribution density of the surface silica layer 3A (3B) in the outer circumferential surface 1a (the inner circumferential surface 1b) of the medical device tube 1, an appropriate distribution density is used, at which sliding properties with respect to a member that comes in contact with the medical device tube 1 when it is used is favorable.

For example, the distribution density of the surface silica layer 3A (3B) in the outer circumferential surface 1a (the inner circumferential surface 1b) may be 70% or more and 100% or less in terms of an area proportion of an area of the surface-exposed silica particles 4a with respect to a surface area of the outer circumferential surface 1a (the inner circumferential surface 1b). For example, an area proportion representing a distribution density of the surface silica layer 3A (3B) in the outer circumferential surface 1a (the inner circumferential surface 1b) may be 90% or more and 100% or less.

Regarding the silica particles 4, appropriate granular silica that forms the surface silica layers 3A and 3B and thus can improve sliding properties of the surface of the medical device tube 1 is used.

The silica particles 4 that are capable of being easily mixed with a polymer oil to be described below are more preferable. Examples of granular silica that easily mixes with a polymer oil to be described below include synthetic amorphous silica.

Examples of synthetic amorphous silica include dry silica purified by a dry method, silica fume, wet silica purified by a wet method, and silica gel. Among these, dry silica is particularly suitable for the silica particles 4 because it is particularly easily mixed with a polymer oil.

The average particle size of the silica particles 4 may be 30 μm or more and 200 μm or less. The average particle size of the silica particles 4 may be 80 μm or more and 110 μm or less.

Next, among the additive components described above, a crosslinking agent, a co-crosslinking agent, and fillers will be described in detail.

Regarding the crosslinking agent, an appropriate crosslinking agent necessary for forming the crosslinked fluorine-based elastomer 2A is selected according to a crosslinking reactive group of the raw materials of the crosslinked fluorine-based elastomer 2A.

For example, regarding the crosslinking agent, an organic peroxide may be used. Specific examples of organic peroxides include ketone peroxides, diacyl peroxides, peroxyketals, alkyl peresters, and percarbonates. Regarding the crosslinking agent, if diacyl peroxides such as 2,5-dimethyl-2,5-di(t-butylperoxy)hexane are used, the reaction is easily initiated since it is readily uniformly distributed.

Regarding the co-crosslinking agent, for example, an organic compound having co-crosslinking reactivity may be used. Examples of an organic compound having co-crosslinking reactivity include triallyl isocyanurate, triallyl cyanurate, triallyl trimerylate, N,N′-m-phenylene dimaleimide, and trimethylolpropane trimethacrylate.

In addition, examples of an organic compound having co-crosslinking reactivity include acrylate or methacrylate monomers.

Among the above exemplified organic compounds having co-crosslinking reactivity, triallyl isocyanurate is particularly preferable.

The co-crosslinking agent in the elastomer layer 2 is contained in an appropriate amount according to the needs of a crosslinking reaction for forming the crosslinked fluorine-based elastomer 2A.

When a crosslinking agent and a co-crosslinking agent are contained in the elastomer layer 2, the content of each of the crosslinking agent and the co-crosslinking agent may be greater than 0 parts by mass and 15 parts by mass or less with respect to 100 parts by mass of the crosslinked fluorine-based elastomer 2A. When the content of the crosslinking agent (co-crosslinking agent) exceeds 15 parts by mass, a crosslinking density of the crosslinked fluorine-based elastomer 2A is excessive, and thus the flexibility of the medical device tube 1 may be impaired.

For example, fillers may be added to reinforce or color the elastomer layer 2. When the elastomer layer 2 is reinforced with fillers, mechanical properties such as the strength of the medical device tube 1 are improved.

Examples of fillers include carbon black and inorganic fillers. Regarding the fillers, a plurality of fillers of different types may be used. For example, regarding the fillers, carbon black and inorganic fillers may be used in combination.

Examples of carbon black include super abrasion furnace (SAF), high abrasion furnace (HAF), semi-reinforcing furnace (SRF), medium thermal (MT), and fast extruding furnace (FEF) carbon blacks. Regarding the carbon black, among these, MT and FEF carbon blacks are particularly preferable. Regarding the carbon black, a plurality of types of carbon black may be used.

Examples of inorganic fillers include barium sulfate, titanium oxide, aluminum oxide, calcium carbonate, calcium silicate, magnesium silicate, and aluminum silicate.

When fillers are contained in the elastomer layer 2, the content of fillers may be greater than 0 parts by mass and 30 parts by mass or less with respect to 100 parts by mass of the crosslinked fluorine-based elastomer 2A.

When the content of fillers exceeds 30 parts by mass, the flexibility of the elastomer layer 2 may be impaired.

The medical device tube 1 is manufactured by a method of manufacturing of an elastomer molded body for a medical device according to the present embodiment.

The method of manufacturing of an elastomer molded body for a medical device according to the present embodiment includes a kneading process, a molding process, and a crosslinking process.

The kneading process includes kneading an elastomer molding material containing a first fluorine-based elastomer, a second fluorine-based elastomer, silica particles 4, and a polymer oil to form a kneaded material.

The first fluorine-based elastomer is a material from which the crosslinked fluorine-based elastomer 2A is formed according to a crosslinking reaction. The first fluorine-based elastomer is formed of the above polymeric fluorine compound as a raw material of the crosslinked fluorine-based elastomer 2A.

The second fluorine-based elastomer is composed of the liquid fluorine-based elastomer 2B in a liquid state.

The silica particles 4 are used only in an amount obtained by adding an amount which can remain inside the elastomer layer 2 of the internal silica particles 4b to an amount necessary for forming the surface silica layers 3A and 3B having a required distribution density and layer thickness. An amount which can remain inside the elastomer layer 2 of the internal silica particles 4b can be checked in advance by experiment or the like.

Regarding the polymer oil, a material having a boiling point equal to or lower than a heating temperature (hereinafter referred to as a “crosslinking heating temperature”) for crosslinking in the crosslinking process to be described below is used. The crosslinking heating temperature is a temperature equal to or higher than a crosslinking temperature that is determined according to a material composition of the first fluorine-based elastomer. In crosslinking of the first fluorine-based elastomer, when a plurality of crosslinking reactions with different crosslinking temperatures are required, the boiling point of the polymer oil may be equal to or lower than the crosslinking heating temperature in crosslinking that proceeds at the lowest temperature. For example, in the crosslinking process, when the crosslinking heating temperature is changed to cause primary (1st order) to nth order crosslinking (here, n is an integer of 2 or more), the boiling point of the polymer oil may be equal to or lower than a crosslinking heating temperature in the primary crosslinking. The boiling point of the polymer oil may be equal to or lower than the crosslinking temperature necessary for a crosslinking reaction in the primary crosslinking.

In addition, the polymer oil needs to be a material that can adhere to at least each of the silica particles 4. The polymer oil may be a material that covers each of the silica particles 4 and can adhere to the silica particles 4.

For example, regarding the polymer oil, a material into which the silica particles 4 are capable of being easily mixed when the silica particles 4 are put into the polymer oil may be selected. Here, for example, the silica particles 4 capable of being easily mixed into a material means that they have high affinity with the polymer oil.

20 parts by mass of the polymer oil may be added with respect to 10 parts by mass of the silica particles 4. 10 parts by mass of the polymer oil may be added with respect to 4.5 parts by mass of the silica particles 4.

In the kneading process, an elastomer molding material is kneaded to form a kneaded material. Regarding the elastomer molding material, the above additive components may be included as necessary in addition to the first fluorine-based elastomer, the second fluorine-based elastomer, the silica particles 4, and the polymer oil described above.

The kneading device and kneading order for the elastomer molding material are not particularly limited as long as the polymer oil can adhere to the silica particles 4.

Regarding the kneading device, a kneading machine, for example, a biaxial roller, a kneader, or a Banbury mixer, may be used.

After the kneading process is performed, the molding process is performed. The molding process includes molding a kneaded material using a mold.

The molding process is performed by an appropriate molding method used for molding an elastomer. Regarding the molding method, for example, press molding, transfer molding, injection molding, or extrusion molding may be used. In order to perform such a molding method, a press molding machine, a transfer molding machine, an injection molding machine, or an extrusion molding machine on which a mold for forming the shape of the medical device tube 1 is mounted is used.

For shaping of the medical device tube 1 according to the present embodiment, for example, an injection molding machine, a transfer molding machine, and an extrusion molding machine may be used.

For example, when the injection molding machine or the transfer molding machine is used, the kneaded material is filled into a molding cavity formed in a tube shape inside the mold mounted in the injection molding machine or the transfer molding machine.

For example, when the extrusion molding machine is used, the kneaded material is continuously extruded in a tube shape from the extrusion mold mounted on the extrusion molding machine.

In this manner, the kneaded material is molded into the same outer shape as the medical device tube 1. In the following, the molded kneaded material is referred to as a molded product.

Thus, the molding process is completed.

After the molding process, the crosslinking process is performed. The crosslinking process includes crosslinking a first fluorine-based elastomer in the molded product by heating a molded product to a boiling point of a polymer oil or higher.

That is, in the crosslinking process, the molded product is heated at a crosslinking heating temperature which is equal to or higher than a boiling point of a polymer oil and equal to or higher than a crosslinking temperature at which crosslinking of a first fluorine-based elastomer proceeds. The crosslinking temperature is a temperature at which the crosslinking reaction starts and a temperature which is determined according to the crosslinking reaction. In the crosslinking process, the crosslinking heating temperature may be a constant temperature or may be changed stepwise in order to control the progress of the crosslinking reaction.

However, the crosslinking process may include crosslinking without heating, for example, radiation crosslinking. When the crosslinking without heating is included in the crosslinking process, a part of the molded product may be crosslinked with heating before the rest of the molded product is crosslinked without heating. In the crosslinking process, when the molded product is crosslinked without heating first, a crosslinking rate of the crosslinking without heating may be 0% or more and less than 50%.

For example, when the injection molding machine is used in the molding process, a molded product formed by being filled into a molding cavity of the mold may remain in the molding cavity without being demolded until the crosslinking process is completed. However, the molded product may be partly crosslinked when remaining in the molding cavity, and, after demolding, may be subjected to secondary (2nd order) or higher order crosslinking by additionally heating at a high temperature using, for example, an oven.

The mold may be heated to a crosslinking temperature or lower before the kneaded material is filled. However, until the kneaded material is completely filled, the temperature of the mold may be lower than the boiling point of the polymer oil in the kneaded material.

When the injection molding machine is used in the molding process, at least a part of the crosslinking process is performed when the kneaded material is completely filled into the mold, the molded product is formed, and the mold is then heated to a crosslinking heating temperature. According to heat conduction from the mold, the first fluorine-based elastomer in the mold is crosslinked according to a crosslinking heating temperature.

Also, when the injection molding machine is used in the molding process, the temperature of the mold changes stepwise, and thus primary crosslinking to nth order crosslinking may proceed.

In the molding process, for example, also when the transfer molding machine or the extrusion molding machine is used, the crosslinking process is performed in the same manner as when the injection molding machine is used.

In the crosslinking process, when the molded product is completely crosslinked, the medical device tube 1 as an elastomer molded body for a medical device is obtained.

Here, the process of forming the surface silica layers 3A and 3B in the above method of manufacturing will be described.

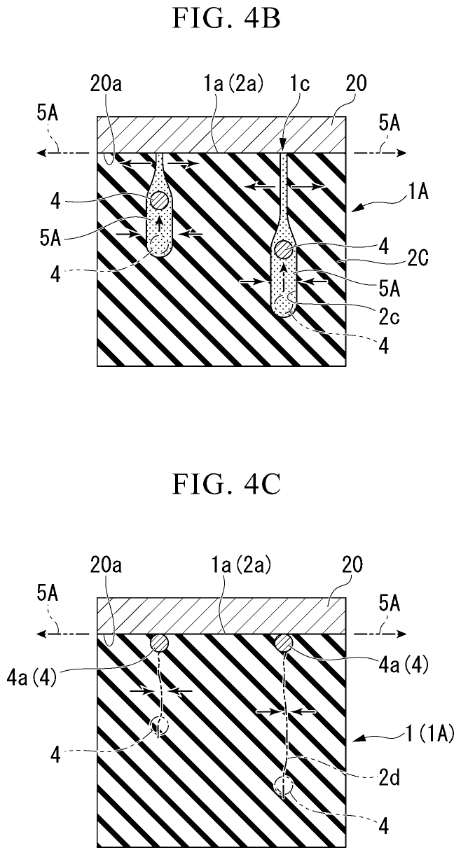

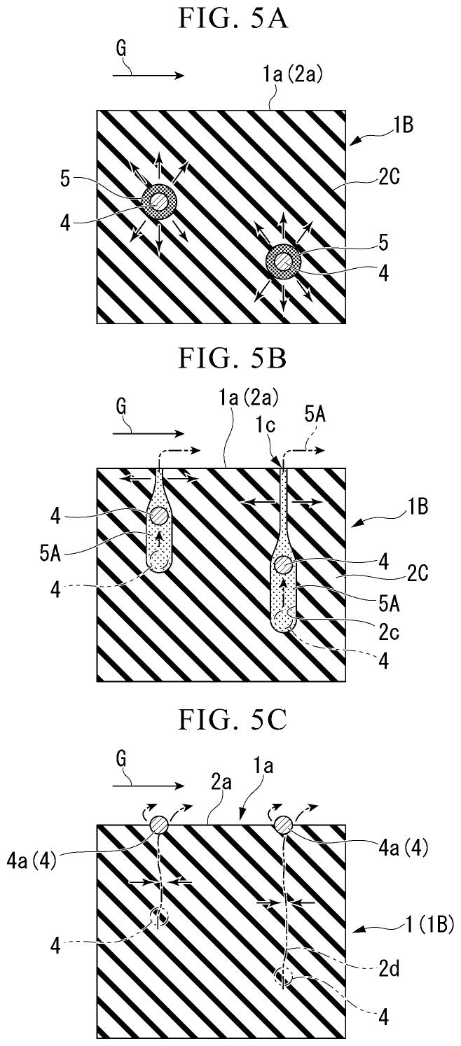

FIG. 4A, FIG. 4B, and FIG. 4C are schematic views showing an example of a manufacturing process of an elastomer molded body for a medical device according to the first embodiment of the present invention. FIG. 5A, FIG. 5B, and FIG. 5C are schematic views showing an example of a manufacturing process of an elastomer molded body for a medical device according to the first embodiment of the present invention.

First, an example (hereinafter referred to as a “mold heating treatment”) in which a molded product can be heated according to heating of the mold using, for example, the injection molding machine or the transfer molding machine will be described.

FIG. 4A schematically shows an appearance in the vicinity of the outer circumferential surface 1a when the kneaded material is introduced into a molding cavity of a mold 20, and a molded product 1A is formed.

The outer circumferential surface 1a of the molded product 1A adheres to a molding surface 20a of the mold 20 and thus the shape of the molding surface 20a is transferred.

As shown in FIG. 4A, the molded product 1A is formed of a mixture containing a fluorine-based elastomer mixture 2C, silica particles 4, and a polymer oil 5.

Here, the fluorine-based elastomer mixture 2C is a mixture of a first fluorine-based elastomer which is uncrosslinked or has not been completely crosslinked and a second fluorine-based elastomer. The fluorine-based elastomer mixture 2C has a certain degree of fluidity depending on the progress of the crosslinking reaction. In the fluorine-based elastomer mixture 2C, particularly, a distribution area of the second fluorine-based elastomer has excellent fluidity.

In the kneading process, the silica particles 4 are dispersed in the fluorine-based elastomer mixture 2C while the polymer oil 5 is adhered.

The polymer oil 5 may contain a component that is dispersed in the fluorine-based elastomer mixture 2C without being adhered to the silica particles 4. However, in FIG. 4A, FIG. 4B, and FIG. 4C, for ease of understanding, the polymer oil 5 which is not adhered to the silica particles 4 is not shown. Similarly, in FIG. 4A, FIG. 4B, and FIG. 4C, the additive components are not shown.

As shown in FIG. 4B, when the mold 20 is heated and thus the temperature of the fluorine-based elastomer mixture 2C becomes equal to or higher than a boiling point of the polymer oil 5, the polymer oil 5 is vaporized and a polymer oil gas 5A is filled around each of the silica particles 4. Thus bubble holes 2c are formed around the silica particles 4.

Since the molded product 1A has a certain degree of fluidity, the polymer oil gas 5A expands toward a softer part in the fluorine-based elastomer mixture 2C. For example, since the molding surface 20a near the heating source has a higher temperature than the inside of the fluorine-based elastomer mixture 2C, the fluorine-based elastomer mixture 2C in the vicinity of the molding surface 20a is particularly soft.

When expansion of the polymer oil gas 5A continues, the bubble holes 2c grow. When an end of the bubble holes 2c reach the outer circumferential surface 1a, the polymer oil gas 5A leaks to the outside of the outer circumferential surface 1a, and openings 1c are formed. The polymer oil gas 5A leaks from the openings 1c to the outside of the fluorine-based elastomer mixture 2C between the outer circumferential surface 1a and the molding surface 20a.

Since the polymer oil gas 5A flows toward each of the openings 1c inside such bubble holes 2c, the silica particles 4 are pushed out toward the openings 1c. When the silica particles 4 move, the bubble holes 2c are blocked by the silica particles 4, and pressure at which the silica particles 4 are pushed out toward the openings 1c become stronger.

The bubble holes 2c after the silica particles 4 have moved are gradually crushed by the pressure surrounding the fluorine-based elastomer mixture 2C.

As shown in FIG. 4C, the silica particles 4 move until they come in contact with the molding surface 20a. From the periphery of the silica particles 4 in contact with the molding surface 20a, the polymer oil gas 5A continues to leak until there is no leaking polymer oil gas 5A. At least some of the silica particles 4 are exposed from the outer circumferential surface 2a of the fluorine-based elastomer mixture 2C. Thereby, the surface-exposed silica particles 4a are realized.

When almost all of the polymer oil gas 5A in the bubble holes 2c leaks to the outside, a joining portion 2d in which the inner walls of each of the bubble holes 2c in which the polymer oil gas 5A had leaked adhere to each other is formed. Thereby, the bubble holes 2c disappear.

In the joining portion 2d, the first fluorine-based elastomers in contact with each other are crosslinked in the crosslinking process and thus integrated. Therefore, in the medical device tube 1 after crosslinking of the molded product 1A sufficiently proceeds, the trace of the joining portion 2d does not remain as cracks or the like.

However, until the crosslinking is completed, the joining portion 2d communicates with another of the bubble holes 2c or another joining portion 2d, and thus can form a leakage path of the polymer oil gas 5A from there.

In this manner, according to the mold heating treatment, when the temperature of the molded product 1A exceeds a boiling point of the polymer oil 5, leaking of the polymer oil 5 from the molded product 1A and movement of the silica particles 4 to the outer circumferential surface 1a start.

The surface-exposed silica particles 4a moved to the outer circumferential surface 1a form the dispersedly-distributed silica layer 3c at the beginning. In the dispersedly-distributed silica layer 3c, when the number of surface-exposed silica particles 4a is further increased, the single layer dense silica layer 3a is formed. When other silica particles 4 move to a part in which the single layer dense silica layer 3a is formed, the multi-layered dense silica layer 3b is formed.

Until the crosslinking is completed, the silica particles 4 that did not move to the outer circumferential surface 1a remain inside the molded product 1A as the internal silica particles 4b.

In this manner, the surface silica layer 3A is formed on the outer circumferential surface 1a.

On the inner circumferential surface 1b of the molded product 1A, similarly, the surface silica layer 3B is formed on the inner circumferential surface 1b.

Such movement of the silica particles 4 starts when the temperature of the molded product 1A exceeds a boiling point of the polymer oil 5. In the crosslinking process, when heating is performed at a higher temperature, movement of the silica particles 4 is further promoted. However, in the crosslinking process, since crosslinking of the first fluorine-based elastomer proceeds, movement of the silica particles 4 becomes gradually slower. Therefore, during the molding process, or until the beginning of the crosslinking process, the heating temperature may be adjusted to an extent that movement of most of the silica particles 4 forming the surface silica layers 3A and 3B is completed. For example, in the crosslinking process, when the crosslinking heating temperature changes stepwise, the surface silica layers 3A and 3B may be formed while heating is performed at a low temperature in the primary crosslinking.

Next, an example (hereinafter referred to as a “direct heating treatment”) when a heating unit other than the mold is required, for example, using an extrusion molding machine, will be described focusing on differences from the mold heating treatment.

FIG. 5A schematically shows an appearance in the vicinity of the outer circumferential surface 1a when a kneaded material is extruded from an extrusion mold (not shown) and a molded product 1B is formed.

As shown in FIG. 5A, the shape of the molding surface of the extrusion mold is transferred to the outer circumferential surface 1a of the molded product 1B. However, the outer circumferential surface 1a is exposed to the outside air.

The configuration of the molded product 1B is the same as that of the molded product 1A. However, in FIG. 5A, FIG. 5B, and FIG. 5C, for ease of understanding, as in FIG. 4A, FIG. 4B, and FIG. 4C, for example, a part of the polymer oil 5, additive components, and the like are not shown.

As shown in FIG. 5A, in the molded product 1B, when the ambient temperature is raised, heating necessary for moving the silica particles 4 and crosslinking is performed. For example, heating of the molded product 1B may be performed when a heating gas G flows along the outer circumferential surface 1a. In order to perform heating from the inner circumferential surface 1b (not shown), for example, the same heating gas G may be caused to flow inside the inner circumferential surface 1b. In the following, an example in which a heating gas G is used to heat the molded product 1B will be described.

As shown in FIG. 5B, when the molded product 1B is heated with the heating gas G, the same bubble holes 2c as in the mold heating treatment are formed.

When heating with the heating gas G proceeds, as in the mold heating treatment, the bubble holes 2c grow, and the openings 1c are formed in the outer circumferential surface 1a.

In the direct heating treatment, since the openings 1c are opened to the outside, the polymer oil gas 5A is more easily leaked from the openings 1c compared to the mold heating treatment. In addition, when a heating gas G flows along the outer circumferential surface 1a, the polymer oil gas 5A leaked from the openings 1c are quickly released from the openings 1c to the outside due to the flow of the heating gas G.

In addition, the silica particles 4 more quickly move because a flow rate of the polymer oil gas 5A leaked from the openings 1c increase.

As shown in FIG. 5C, at least some of the silica particles 4 are exposed from the outer circumferential surface 2a, and the surface-exposed silica particles 4a are realized. The surface-exposed silica particles 4a adhere to the surrounding fluorine-based elastomer mixture 2C after the polymer oil gas 5A has passed and thus fixed to the outer circumferential surface 1a (2a).

When almost all of the polymer oil gas 5A leaks to the outside, the same joining portion 2d as in the mold heating treatment is formed. Thereby, the bubble holes 2c disappear.

As in the mold heating treatment, in the joining portion 2d, the first fluorine-based elastomers in contact with each other are crosslinked in the crosslinking process and thus integrated.

In this manner, in the direct heating treatment, the polymer oil 5 is leaked from the molded product 1B, and the silica particles 4 move to the outer circumferential surface 1a in the same manner as in the mold heating treatment except that the polymer oil gas 5A easily leaks to the outside through the openings 1c that are open to the outside.

Therefore, also in the direct heating treatment, as in the mold heating treatment, the surface silica layer 3A is formed on the outer circumferential surface 1a, and the surface silica layer 3B is formed on the inner circumferential surface 1b.

As described above, in the method of manufacturing of an elastomer molded body for a medical device according to the present embodiment, the silica particles 4 dispersed inside the molded product 1A (1B) move to the outer circumferential surface 1a and the inner circumferential surface 1b together with the polymer oil gas 5A according to heating of the molded product 1A (1B). Therefore, most of the silica particles 4 dispersed inside the molded product 1A (1B) are distributed to the surface layer part Ss. The surface silica layers 3A and 3B are formed on the surface of the medical device tube 1.

Since the medical device tube 1 manufactured in this manner has the surface silica layers 3A and 3B, sliding properties of the outer circumferential surface 1a and the inner circumferential surface 1b are improved compared to when the surface is formed of only the elastomer layer 2. Therefore, sliding properties with respect to the member that comes in contact with the medical device tube 1 when it is used become favorable.

The layer thickness of the surface silica layers 3A and 3B can be adjusted according to an amount of the silica particles 4 and the polymer oil 5 added to the kneaded material, and movement characteristics of the silica particles 4 in the molded product 1A (1B). When the layer thickness of the surface silica layers 3A and 3B is set to an extent that the flexibility of the medical device tube 1 is not impaired, a decrease in the flexibility of the medical device tube 1 due to the surface silica layers 3A and 3B is limited.

In addition, in the medical device tube 1, since the silica particles 4 are unevenly distributed in the surface layer portion Ss, a distribution of the silica particles 4 in the center portion Sc is reduced to 0% or more and 20% or less. Therefore, the flexibility of the medical device tube 1 is improved compared to when more silica particles 4 are contained in the center portion Sc.

In addition, the elastomer layer 2 of the medical device tube 1 is included in the liquid fluorine-based elastomer 2B. Since the liquid fluorine-based elastomer 2B is not crosslinked with the crosslinked fluorine-based elastomer 2A, the flexibility of the medical device tube 1 is improved compared to when the liquid fluorine-based elastomer 2B is not contained.

In this manner, in the medical device tube 1 according to the present embodiment, sliding properties with respect to the surface are improved while maintaining flexibility. In addition, in the method of manufacturing of the medical device tube 1 according to the present embodiment, according to addition of the polymer oil 5 in the kneaded material, the medical device tube 1 that can improve sliding properties with respect to the surface while maintaining flexibility can be easily manufactured.

### Second Embodiment

A medical device according to a second embodiment of the present invention will be described.

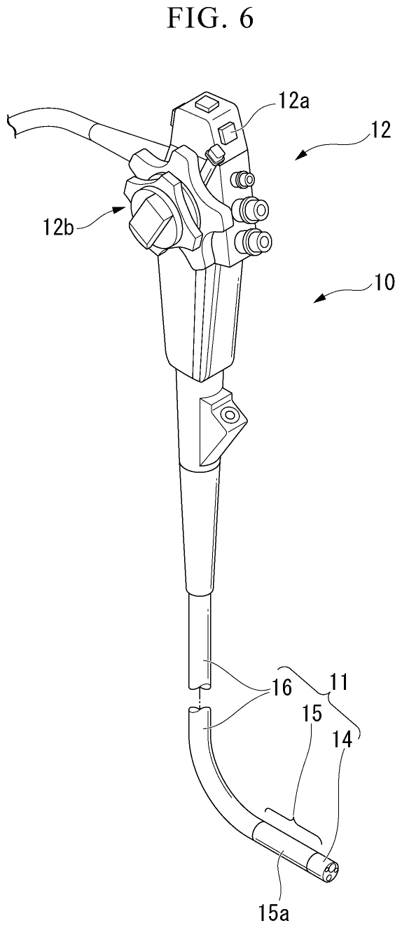

FIG. 6 is a perspective view schematically showing an example of a medical device according to the second embodiment of the present invention.

As shown in FIG. 6, an endoscope 10 (medical device) of the present embodiment includes an insertion portion 11 and an operation unit 12

The insertion portion 11 is formed in a flexible tubular shape so that it can be inserted into a patient's body. In the insertion portion 11, a distal end portion 14, a bending portion 15, and a flexible tube portion 16 are provided in order from the distal end side in the insertion direction. Although not specifically shown, an endoscopic channel through which an endoscopic device passes may be provided inside the insertion portion 11 in the longitudinal direction.

The distal end portion 14 is a portion that is disposed at the distal end portion of the endoscope 10 and has an end effector as a manipulator. In the present embodiment, for example, the distal end portion 14 internally includes an imaging optical system including an imaging element such as a CCD and an appropriate lens in order to acquire an image of a subject, and has a columnar outer shape.

An imaging window and a lighting window are formed at the distal end of the distal end portion 14. When the insertion portion 11 includes an endoscopic channel, an opening of the endoscopic channel is provided at the distal end of the distal end portion 14.

The bending portion 15 is connected to the proximal end side of the distal end portion 14. The bending portion 15 is bendable in order to change the direction of the distal end portion 14. The bending portion 15 is a tubular part.

For example, the bending portion 15 is configured by connecting a plurality of annular joint rings that are rotatable, and a plurality of angle wires are inserted thereinto.

For example, members such as an electrical wiring connected to an imaging element of the distal end portion 14 and a light guide that extends to a lighting window are accommodated inside the bending portion 15. These members such as an electrical wiring and a light guide are inserted into the flexible tube portion 16 to be described below and extend to the operation unit 12 to be described below.

The bending portion 15 is covered by an outer tube 15a (elastomer molded body for a medical device).

For the outer tube 15a, the same configuration as that of the medical device tube 1 according to the first embodiment is used.

The flexible tube portion 16 is a tubular portion that connects the bending portion 15 to the operation unit 12 to be described below.

The flexible tube portion 16 includes, for example, a spiral tube in which a metal or resin band-like member is spirally wound and a soft covering resin. The covering resin covers the outer periphery of the spiral tube in a tubular shape.

In such a configuration, the flexible tube portion 16 can be bent in an appropriate direction while maintaining a substantially circular cross section.

The material of the covering resin in the flexible tube portion 16 is not particularly limited. For example, regarding the covering resin in the flexible tube portion 16, the same configuration as that of the medical device tube 1 according to the first embodiment may be used.

A coil sheath is disposed inside the flexible tube portion 16, and angle wires extending from the bending portion 15 to the proximal end side are inserted into the coil sheath. As in the bending portion 15, members such as the above electrical wiring and light guide are inserted into the flexible tube portion 16.

The operation unit 12 is a device unit through which an operator performs an operation of the endoscope 10. Examples of an operation that is performed through the operation unit 12 include an operation of pulling angle wires in order to change a bending amount of the bending portion 15. The operation unit 12 includes, for example, an operation switch 12a and an operation knob 12b.

For example, the operation switch 12a is composed of a switch button.

In the operation switch 12a, materials of a button body that is exposed from the operation unit 12 or an outer cover that covers the button body are not particularly limited. Regarding the button body of the operation switch 12a or the outer cover that covers the button body, the elastomer molded body for a medical device according to the first embodiment formed into each shape may be used.

Although not specifically shown, for example, an O-ring, a sealing member, and the like are disposed inside the insertion portion 11. Regarding the O-ring and the sealing member which are not shown, the elastomer molded body for a medical device according to the first embodiment formed into each shape may be used.

For example, the endoscope 10 of the present embodiment has the same configuration as that of the elastomer molded body for a medical device according to the first embodiment such as the outer tube 15a. Therefore, the endoscope 10 has the same function as the elastomer molded body for a medical device according to the first embodiment.

For example, in the outer tube 15a, when the bending portion 15 is bent, since sliding properties with respect to the surface are improved while maintaining flexibility, a bending load can be reduced. For example, since sliding properties between the inner circumferential surface of the outer tube 15a and an internal member such as a spiral tube in contact with the inner circumferential surface become favorable, a sliding load is reduced. For example, since sliding properties between the outer circumferential surface of the outer tube 15a and another medical device that is disposed outside the bending portion 15 become favorable, a sliding load is reduced.

Here, in the description of the embodiments, an example in which the elastomer included in the elastomer layer 2 of the elastomer molded body for a medical device is only a fluorine-based elastomer has been described. However, an elastomer other than the fluorine-based elastomer may be included in the elastomer layer 2.

For example, examples of elastomers other than the fluorine-based elastomer include ethylene propylene diene rubber (EPDM), ethylene propylene rubber (EPT), and a silicone elastomer.

### EXAMPLES

Examples of the medical device tube 1 according to the first embodiment will be described below together with comparative examples. In the following [Table 1], the composition and manufacturing conditions of medical device tubes (in [Table 1], described as a “molded body”) of Examples 1 to 8, and Comparative Examples 1 to 4 are shown. Here, in [Table 1], reference numerals are not shown.

TABLE 1

| | COMPOSITION OF MOLDED BODY (PARTS BY MASS) | | | | | | |
|---|---|---|---|---|---|---|---|
| | CROSSLINKED | LIQUID | SILICA PARTICLES | | | | |
| | FLUORINE-BASED | FLUORINE-BASED | DRY | WET | CROSSLINKING | CO-CROSSLINKING | |
| | ELASTOMER | ELASTOMER | SILICA | SILICA | AGENT | AGENT | FILLER |
| EXAMPLE 1 | 100 | 20 | 3 | 0 | 0.7 | 0 | 0 |
| EXAMPLE 2 | 100 | 20 | 3 | 0 | 0.7 | 6 | 0 |
| EXAMPLE 3 | 100 | 20 | 3 | 0 | 0.7 | 16 | 0 |
| EXAMPLE 4 | 100 | 20 | 3 | 0 | 0.7 | 0 | 20 |
| EXAMPLE 5 | 100 | 20 | 3 | 0 | 0.7 | 0 | 40 |
| EXAMPLE 6 | 100 | 20 | 3 | 0 | 0.7 | 0 | 0 |
| EXAMPLE 7 | 100 | 20 | 0 | 3 | 0.7 | 0 | 0 |
| EXAMPLE 8 | 100 | 20 | 4.5 | 0 | 0.7 | 0 | 0 |
| COMPARATIVE | 100 | 20 | 0 | 0 | 0.7 | 0 | 0 |
| EXAMPLE 1 | | | | | | | |
| COMPARATIVE | 100 | 20 | 10 | 0 | 0.7 | 0 | 0 |
| EXAMPLE 2 | | | | | | | |
| COMPARATIVE | 100 | 20 | 3 | 0 | 0.7 | 0 | 0 |
| EXAMPLE 3 | | | | | | | |
| COMPARATIVE | 100 | 20 | 3 | 0 | 0.7 | 0 | 0 |
| EXAMPLE 4 | | | | | | | |

| | MANUFACTURING CONDITIONS | | |
|---|---|---|---|
| | POLYMER OIL | | PRIMARY |
| | AMOUNT ADDED | BOILING | CROSSLINKING |
| | (PARTS BY | POINT | TEMPERATURE |
| | MASS) | (° C.) | (° C.) |
| EXAMPLE 1 | 10 | 153 | 160 |
| EXAMPLE 2 | 10 | 153 | 160 |
| EXAMPLE 3 | 10 | 153 | 160 |
| EXAMPLE 4 | 10 | 153 | 160 |
| EXAMPLE 5 | 10 | 153 | 160 |
| EXAMPLE 6 | 10 | 153 | 165 |
| EXAMPLE 7 | 10 | 153 | 160 |
| EXAMPLE 8 | 10 | 153 | 160 |
| COMPARATIVE | 10 | 153 | 160 |
| EXAMPLE 1 | | | |
| COMPARATIVE | 0 | — | 160 |
| EXAMPLE 2 | | | |
| COMPARATIVE | 10 | 220 | 160 |
| EXAMPLE 3 | | | |
| COMPARATIVE | 10 | 153 | 150 |
| EXAMPLE 4 | | | |

### Example 1

As shown in the above [Table 1], the composition of the medical device tube 1 of Example 1 included 100 parts by mass of the crosslinked fluorine-based elastomer 2A, 20 parts by mass of the liquid fluorine-based elastomer 2B, 3 parts by mass of the silica particles 4, and 0.7 parts by mass of the crosslinking agent.

Regarding the crosslinked fluorine-based elastomer 2A, a crosslinked fluorine rubber containing a vinylidene fluoride-hexafluoropropylene-tetrafluoroethylene copolymer as a main component was used.

Regarding the liquid fluorine-based elastomer 2B, a liquid fluorine rubber containing a vinylidene fluoride-hexafluoropropylene copolymer as a main component was used.

Regarding the silica particles 4, dry silica was used.

Regarding the crosslinking agent, 2,5-dimethyl-2,5-bis(tert-butylperoxy)hexane as an organic peroxide was used.

Example 1 did not contain co-crosslinking agent and filler.

An evaluation sample of the medical device tube 1 of Example 1 was manufactured using the method of manufacturing of an elastomer molded body for a medical device according to the first embodiment described above. The shape of the evaluation sample was a cylindrical tube having an outer diameter of 12 mm, a wall thickness of 0.5 mm, and a length of 15 mm.

In the kneading process, the polymer oil 5 was added to the above crosslinked fluorine rubber raw material (first fluorine-based elastomer), liquid fluorine rubber raw material (second fluorine-based elastomer), dry silica, and crosslinking agent, and these materials were kneaded with an open roller. Thereby, a molding material compound (kneaded material) was formed.

Here, regarding the polymer oil 5, 10 parts by mass of a polymer oil having a boiling point of 153° C. and capable of being easily mixed with the silica particles 4 was used.

In the molding process, the kneaded material was filled into the molding cavity of the mold by the transfer molding machine. The molding cavity of the mold was formed in a shape corresponding to the shape of the evaluation sample. The shape of the molding surface of the mold is transferred to the kneaded material filled into the molding cavity of the mold. Thereby, a molded product having an outer shape of the evaluation sample was formed inside the mold.

In the crosslinking process, primary crosslinking and secondary crosslinking were caused to proceed.

The primary crosslinking was caused to proceed by heating the mold. In this example, the crosslinking temperature for the crosslinking reaction in the primary crosslinking was 158° C. A crosslinking heating temperature for primary crosslinking (in [Table 1], described as “primary crosslinking temperature”) was 160° C. A heating time for primary crosslinking was 3 minutes.

The secondary crosslinking was performed when the molded product after the primary crosslinking was removed from the mold and the molded product was moved into an oven. The crosslinking heating temperature for the secondary crosslinking was 180° C., and a heating time was 4 hours.

After the secondary crosslinking was completed, an evaluation sample of the medical device tube 1 of Example 1 was obtained.

### Examples 2 and 3

The medical device tube 1 of Example 2 was different from that of Example 1 in that 6 parts by mass of a co-crosslinking agent was added. Regarding the co-crosslinking agent, triallyl isocyanurate was used.

An evaluation sample of the medical device tube 1 of Example 2 was manufactured in the same manner as in Example 1 except that a co-crosslinking agent was added to the kneaded material.

The medical device tube 1 of Example 3 was different from that of Example 2 in that 16 parts by mass of a co-crosslinking agent was added.

### Examples 4 and 5

The medical device tube 1 of Example 4 was different from that of Example 1 in that 20 parts by mass of a filler was added. Regarding the filler, MT carbon as carbon black was used.

An evaluation sample of the medical device tube 1 of Example 4 was manufactured in the same manner as in Example 1 except that a filler was added to the kneaded material.

The medical device tube 1 of Example 5 was different from that of Example 4 in that 40 parts by mass of a filler was added.

### Example 6

The composition of the medical device tube 1 of Example 6 was the same as that of Example 1.

An evaluation sample of the medical device tube 1 of Example 6 was manufactured in the same manner as in Example 1 except that the primary crosslinking temperature (primary crosslinking heating temperature) was 165° C.

### Example 7

The medical device tube 1 of Example 7 was different from that of Example 1 in that 3 parts by mass of wet silica was used as the silica particles 4.

An evaluation sample of the medical device tube 1 of Example 7 was manufactured in the same manner as in Example 1 except that wet silica was added to the kneaded material.

### Example 8

The medical device tube 1 of Example 8 was different from that of Example 1 in that 4.5 parts by mass of dry silica was added.

An evaluation sample of the medical device tube 1 of Example 8 was manufactured in the same manner as in Example 1 except that an amount of dry silica added to the kneaded material was changed.

### Comparative Example 1

As shown in the above [Table 1], a medical device tube of Comparative Example 1 was formed in the same manner as in Example 1 except that no silica particles were contained.

An evaluation sample of the medical device tube of Comparative Example 1 was manufactured in the same manner as in Example 1 except that no silica particles were added to the kneaded material.

### Comparative Example 2

A medical device tube of Comparative Example 2 was different from that of Example 1 in that the content of dry silica was 10 parts by mass.

An evaluation sample of the medical device tube of Comparative Example 2 was manufactured in the same manner as in Example 1 except that an amount of dry silica added to the kneaded material was changed and no polymer oil was added.

### Comparative Examples 3 and 4

A medical device tube of Comparative Example 3 was manufactured in the same manner as in Example 1 except that, when an evaluation sample was manufactured, a polymer oil having a boiling point of 220° C. in place of the polymer oil 5 of Example 1 was added to the kneaded material.

A medical device tube of Comparative Example 4 was manufactured in the same manner as in Example 1 except that, when an evaluation sample was manufactured, the primary crosslinking temperature was set to 150° C., which is lower than the boiling point of the polymer oil. However, since the first fluorine-based elastomer was not crosslinked at 150° C., in Comparative Example 4, a temperature of 150° C. did not represent the crosslinking heating temperature. However, since heating for secondary crosslinking was performed as in Example 1, after heating for secondary crosslinking, the evaluation sample of Comparative Example 4 was completely crosslinked.

### [Evaluation]

Regarding evaluation of the elastomer molded bodies for a medical device of the above examples and comparative examples, observation of silica particles on the surfaces (the outer circumferential surface and the inner circumferential surface) of the evaluation samples, evaluation of flexibility, evaluation of sliding properties, evaluation of tear strength, evaluation of breaking strength, and comprehensive evaluation shown in the following [Table 2] were performed.

TABLE 2

| | EVALUATION RESULTS | | | | |
|---|---|---|---|---|---|
| | | | TEAR | BREAKING | |
| | | SLIDING | STRENGTH | STRENGTH | |
| | FLEXIBILITY | PROPERTIES | (N/mm) | (MPa) | COMPREHENSIVE |
| EXAMPLE 1 | A | A | A | A | A |
| EXAMPLE 2 | A | A | AA | — | AA |
| EXAMPLE 3 | B | A | AA | — | B |
| EXAMPLE 4 | A | A | — | AA | AA |
| EXAMPLE 5 | B | A | — | AA | B |
| EXAMPLE 6 | A | AA | — | — | AA |
| EXAMPLE 7 | A | A | — | — | A |
| EXAMPLE 8 | B | AA | — | — | B |
| COMPARATIVE | A | C | — | — | C |
| EXAMPLE 1 | | | | | |
| COMPARATIVE | C | C | — | — | C |
| EXAMPLE 2 | | | | | |
| COMPARATIVE | A | C | — | — | C |
| EXAMPLE 3 | | | | | |
| COMPARATIVE | A | C | — | — | C |
| EXAMPLE 4 | | | | | |

### [Evaluation Method]

In order to observe the surfaces of the evaluation samples, a scanning electron microscope was used. An evaluator evaluated the distribution, the layer thickness, and the like of silica particles based on the image from the scanning electron microscope.

The flexibility was evaluated using the 100% modulus according to the tensile test based on JIS K6251 as an index. Therefore, in addition to the evaluation samples of the tube shape described above, tensile test pieces based on JIS K6251 were produced using the elastomer molded bodies for a medical device of the examples and the comparative examples.

A lower 100% modulus indicated better flexibility.

In evaluation of flexibility, when the 100% modulus was less than 1.3 MPa, the flexibility was evaluated as “good” (in [Table 2], indicated as “A” (good)), when the 100% modulus was 1.3 MPa or more and less than 1.35 MPa, the flexibility was evaluated as “fair” (in [Table 2], indicated as “B” (fair)), and when the 100% modulus was 1.35 MPa or more, the flexibility was evaluated as “no good” (in [Table 2], indicated as “C” (no good)).

The sliding properties were evaluated using a coefficient of dynamic friction according to the test of a coefficient of friction based on JIS K7125 as an index. Therefore, in addition to the evaluation samples of the tube shape described above, test pieces based on JIS K7125 were produced using the elastomer molded bodies for a medical device of the examples and the comparative examples.

A lower coefficient of dynamic friction indicated better sliding properties.

In evaluation of sliding properties, when the coefficient of dynamic friction was less than 0.7, the sliding properties were evaluated as “very good” (in [Table 2], indicated as “AA” (very good)), when the coefficient of dynamic friction was 0.7 or more and less than 0.8, the sliding properties were evaluated as “good” (in [Table 2], indicated as “A” (good)), and when the coefficient of dynamic friction was 0.8 or more, the sliding properties were evaluated as “no good” (in [Table 2], indicated as “C” (no good)).

The tear strength was evaluated using the tear strength according to the tensile test based on JIS K6252 as an index. The tear strengths of Examples 1 to 3 were evaluated.

The higher tear strength was more preferable in consideration of durability.

In evaluation of the tear strength, when the tear strength was 35 N/mm or more, the tear strength was evaluated as “very good” (in [Table 2], indicated as “AA” (very good)), when the tear strength was 30 N/mm or more and less than 35 N/mm, the tear strength was evaluated as “good” (in [Table 2], indicated as “A” (good)), and when the tear strength was less than 30 N/mm, the tear strength was evaluated as “no good” (in [Table 2], indicated as “C” (no good)).

In evaluation of the breaking strength, evaluation was performed using the breaking strength according to the tensile test based on JIS K6251 as an index. The breaking strengths of Examples 1, 4, and 5 were evaluated.

The higher breaking strength was more preferable in consideration of durability.

In evaluation of the breaking strength, when the breaking strength was 20 MPa or more, the breaking strength was evaluated as “very good” (in [Table 2], indicated as “AA” (very good)), when the breaking strength was 15 MPa or more and less than 20 MPa, the breaking strength was evaluated as “good” (in [Table 2], indicated as “A” (good)), and when the breaking strength was less than 15 MPa, the breaking strength was evaluated as “no good” (in [Table 2], indicated as “C” (no good)).

Comprehensive evaluation was performed in four degrees: “very good” (in [Table 2], indicated as “AA” (very good)), “good” (in [Table 2], indicated as “A” (good)), “fair” (in [Table 2], indicated as “B” (fair)), and “no good” (in [Table 2], indicated as “C” (no good)).

When a lower evaluation degree between evaluation of flexibility and evaluation of sliding properties was “no good” or “fair,” the comprehensive evaluation was determined as the lowest evaluation degree between evaluation of flexibility and evaluation of sliding properties.

When “no good” and “fair” were not included in evaluation of flexibility and evaluation of sliding properties, comprehensive evaluation was determined as the highest evaluation degree among all of the evaluations.

### [Evaluation Results]

First, observation results of the evaluation samples will be described.

On the surface of the evaluation sample of Example 1, the surface silica layers 3A and 3B each having a layer thickness of larger than 0 μm and 10 μm or less, and substantially uniformly distributed on the surface were formed. According to observation of the cross section of the evaluation sample, there were no silica particles in the center portion in the layer thickness direction, and the silica particles 4 were unevenly distributed on the surface. Examples 2 to 8 were almost the same as Example 1.

On the other hand, no silica particles were observed on the surface of the evaluation sample of Comparative Example 1 containing no silica particles.

There were silica particles on the surfaces of the evaluation samples of Comparative Examples 2 to 4. However, according to observation of the cross section of the evaluation sample, there were a large number of silica particles in the center portion in the layer thickness direction, and the distribution density of the surface was not higher than that of the center portion.

Accordingly, since the distribution density of silica particles in Comparative Examples 2 to 4 was not higher on the surface of the medical device tube than in the center portion, the distribution density of silica particles on the surface was lower than that of Examples 1 to 8.

In Comparative Example 2, it was thought that, since no polymer oil was contained in the kneaded material, silica particles inside the molded product could not move to the surface.

In all of Comparative Examples 3 and 4, the boiling point of the polymer oil contained in the kneaded material was higher than the primary crosslinking temperature. Therefore, in Comparative Examples 3 and 4, it was thought that the polymer oil was not vaporized during primary crosslinking, and silica particles inside the molded product could not move to the surface.

As shown in [Table 2], in evaluation of flexibility, Examples 1, 2, 4, 6, and 7, and Comparative Examples 1, 3, and 4 were evaluated as “good.” Examples 3, 5, and 8 were evaluated as “fair.” Comparative Example 2 was evaluated as “no good.”

It was thought that Example 3 had lower flexibility than Examples 1 and 2 because the content of the co-crosslinking agent was 16 parts by mass with respect to 100 parts by mass of the crosslinked fluorine-based elastomer. The content of the co-crosslinking agent may be 15 parts by mass or less. However, when the content of the co-crosslinking agent was about 16 parts by mass, the flexibility was not rated as “no good.”

It was thought that Example 5 had lower flexibility than Examples 1 and 4 because the content of the filler was 40 parts by mass with respect to 100 parts by mass of the crosslinked fluorine-based elastomer. The content of the filler may be 30 parts by mass or less. However, when the content of the filler was about 40 parts by mass, the flexibility was not rated as “no good.”

It was thought that Example 8 had lower flexibility than Example 1 because the content of silica particles was 4.5 parts by mass with respect to 100 parts by mass of the crosslinked fluorine-based elastomer. This is thought to be caused by the fact that, when the content of silica particles increased, the surface silica layers 3A and 3B became denser. However, when the content of silica particles was about 4.5 parts by mass, the flexibility was not rated as “no good.”

On the other hand, in Comparative Example 2, since no polymer oil was contained in the kneaded material, silica particles were substantially uniformly dispersed in the layer thickness direction of the evaluation sample. In addition, an amount of silica particles contained in Comparative Example 2 was 10 parts by mass, which was three or more times that of Example 1. Therefore, it was thought that the flexibility was lower than that of Example 1 because the number of silica particles distributed inside the evaluation sample was too large.

It was thought that Comparative Example 1 had favorable flexibility because silica particles were not contained at all.

In Comparative Examples 3 and 4, since the crosslinking heating temperature of primary crosslinking was lower than the boiling point of the polymer oil, silica particles did not move to the surface in the primary crosslinking. Therefore, relatively more silica particles remained inside the evaluation sample than on the surface. However, it was thought that the flexibility was not rated as “no good” because there were silica particles that moved to the surface in secondary crosslinking and the content of silica particles themselves was smaller than that of Comparative Example 1.

In evaluation of sliding properties, Examples 6 and 8 were evaluated as “very good,” and Examples 1 to 5, and 7 were evaluated as “good.” Comparative Examples 1 to 4 were evaluated as “no good.”

It was thought that, since Example 6 had a higher primary crosslinking temperature than Example 1, movement of the silica particles 4 to the surface was promoted. Therefore, it was thought that, in Example 6, the surface silica layers 3A and 3B were formed more densely than those of Example 1, sliding properties were improved.

It was thought that, since Example 8 had a higher content of the silica particles 4 in the kneaded material than Example 1, an amount of the silica particles 4 moved to the surface increased even if the primary crosslinking temperature was the same. Therefore, it was thought that, in Example 8, the surface silica layers 3A and 3B were formed more densely than those of Example 1, and the sliding properties were improved.

On the other hand, it was thought that, in all of Comparative Examples 1 to 4, since the distribution density of silica particles on the surface of the evaluation samples was too low, the sliding properties was rated as “no good.”

In evaluation of the tear strength, Examples 2 and 3 were evaluated as “very good,” and Example 1 was evaluated as “good.” It was thought that, in Examples 2 and 3, the tear strength was improved because crosslinking of the first fluorine-based elastomer was promoted according to addition of the co-crosslinking agent.

In evaluation of the breaking strength, Examples 4 and 5 were evaluated as “very good,” and Example 1 was evaluated as “good.” It was thought that, in Examples 4 and 5, the breaking strength was improved because the elastomer layer 2 was reinforced according to addition of the filler.

In comprehensive evaluation, Examples 2, 4, and 6 were evaluated as “very good,” Examples 1 and 7 were evaluated as “good,” and Examples 3, 5, and 8 were evaluated as “fair.” That is, it was determined that all of Examples 1 to 8 had both flexibility and sliding properties.

On the other hand, all of Comparative Examples 1 to 4 were evaluated as “no good.” Therefore, it was determined that all of Comparative Examples 1 to 4 failed to have both flexibility and sliding properties.

While preferred embodiments of the invention have been described and illustrated above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

What is claimed is:

1. An elastomer molded body for a medical device, comprising:
an elastomer portion containing a fluorine-based elastomer; and
a plurality of silica particles more densely distributed in outside of a center portion of the elastomer portion than inside of the center portion, at least some of the plurality of silica particles being exposed to a surface of the elastomer portion.

2. The elastomer molded body for a medical device according to claim 1,
wherein a silica particle group exposed to the surface of the elastomer portion among the plurality of silica particles is distributed in a layer form having thickness of larger than 0 μm and 10 μm or less on the surface.

3. The elastomer molded body for a medical device according to claim 1,
wherein the fluorine-based elastomer includes
a crosslinked fluorine-based elastomer, and
a liquid fluorine-based elastomer that is not crosslinked with the crosslinked fluorine-based elastomer.

4. The elastomer molded body for a medical device according to claim 3, comprising
a co-crosslinking agent which amount being greater than 0 parts by mass and 15 parts by mass or less with respect to 100 parts by mass of the crosslinked fluorine-based elastomer.

5. The elastomer molded body for a medical device according to claim 3, comprising
a filler which amount being greater than 0 parts by mass and 30 parts by mass or less with respect to 100 parts by mass of the crosslinked fluorine-based elastomer.

6. A medical device comprising the elastomer molded body for a medical device according to claim 1.

7. A method of manufacturing of an elastomer molded body for a medical device, comprising:
kneading an elastomer molding material containing a crosslinkable first fluorine-based elastomer, a second fluorine-based elastomer composed of a liquid fluorine-based elastomer unable to crosslink with the first fluorine-based elastomer, a polymer oil, and a plurality of silica particles, and forming a kneaded material;
molding the kneaded material using a mold; and
heating the molded kneaded material to a temperature that is equal to or higher than a boiling point of the polymer oil to crosslink, the first fluorine-based elastomer in the kneaded material.
